(19) United States
(12) Patent Application Publication
(10) Pub. No.: US 2019/0322638 A1
(43) Pub. Date: Oct. 24, 2019

(54) DIPYRIDYL ALKALOID, PREPARATION METHOD THEREFOR AND USE THEREOF

(71) Applicant: OCEAN UNIVERSITY OF CHINA, Qingdao (CN)

(72) Inventors: Weiming ZHU, Qingdao (CN); Xiangui MEI, Qingdao (CN); Peng FU, Qingdao (CN); Yi WANG, Qingdao (CN); Peipei LIU, Qingdao (CN)

(21) Appl. No.: 16/349,915

(22) PCT Filed: Nov. 14, 2017
(86) PCT No.: PCT/CN2017/110799

§ 371 Date: May 14, 2019

(30) Foreign Application Priority Data
Nov. 15, 2016 (CN) 201611014546.8

(51) Int. Cl. C07D 401/04; A61K 9/19; A61P 35/00; A61K 9/00; C07H 17/02

(52) U.S. Cl. CPC C07D 401/04; A61K 9/19; C07H 17/02; A61K 9/0019; A61P 35/00

(57) ABSTRACT

Disclosed are dipyridyl alkaloid, a preparation method therefor and use thereof. The structure of the dipyridyl alkaloid is as shown in formula I. The dipyridyl alkaloid has a tumor cell proliferation inhibitory activity and can be used as a tumor cell proliferation inhibitor or for developing an anti-tumor drug.

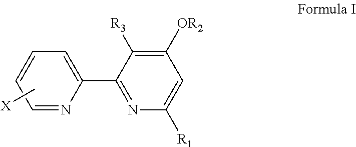

### TECHNICAL FIELD

The present disclosure relates to dipyridyl alkaloid, a preparation method therefor and use thereof.

### BACKGROUND ART

According to the “2015 report on Chinese nutrition and chronic disease”, 533 out of every 100,000 Chinese residents died from chronic disease in 2012, accounting for 86.6 percent of all deaths, with cardia-cerebrovascular disease, cancer and chronic respiratory disease the top causes, accounting for 79.4 percent of all deaths, among which cancer mortality was 144.3 out of every 100,000 people (the top five were lung cancer, liver cancer, gastric cancer, esophagus cancer, and colorectal cancer, respectively). Moreover, as indicated in the report published on “CA: A Cancer journal_for_Clinicians” in 2016, with increasing incidence and mortality, cancer is the leading cause of death for many diseases in China; an estimated 4292,000 new cancer cases and 2814,000 cancer deaths occurred in China in 2015, and almost 22% of global new cancer cases and close to 27% of global cancer deaths occurred in China Cancer has become a major public health problem. Because of China's massive population, these Chinese data contribute significantly to prevention and control of the global cancers (see Chen W, Zheng R, Baade P D, et al. “Cancer statistics in China, 2015”. Ca-Cancer. J. Clin. 2016, 66:115-132. doi:10.3322/caac.21338). In 2011, the article “Cancer crusade at 40” in the special section of Science indicated that nowadays, cancer is still a major life-threatening disease for human, and that the cancer mortality in the United States remains high since 1971 and many types of cancers, such as prostatic cancer, are still incurable (see Kiberstis P, Marshall E. Cancer crusade at 40. Celebrating an anniversary. Introduction. Science 2011, 331: 1539).

As the ever-accelerated updating of science and technology and medical level, humans have explored a variety of methods for treatment of tumors, which primarily fall into surgery, chemotherapy, radiotherapy, multi-disciplinary comprehensive treatment integrated with traditional Chinese medicine, and the like (see Shengxiang YAN, Huaping YUAN. “Discussing on Therapies of Tumors”, Journal of Chinese Physician. 2002 (supplement), 55). Among the methods, chemotherapy is one of the important means to treat cancer patients. Chemotherapy is a therapy method of killing tumor cells, inhibiting growth and proliferation of tumor cells, and promoting differentiation of tumor cells by virtue of chemical drugs. However, because of poor selectivity of chemotherapy, chemotherapeutic drugs kill a large number of normal cells and immune cells, while killing tumor cells, they have strong side effects and toxic effects. Chemotherapy may cause adverse effects, such as damage of hepatorenal functions, gastrointestinal disorders, decreased immunological function, alopecia, nausea and vomiting. Almost all chemotherapeutic drugs cause liver function damage. In mild cases, abnormal liver function may occur, and patients may feel unwell in hepatic region, while toxic hepatitis may be incurred in severe cases. Some chemotherapeutic drugs when administered at a large dose may cause renal function damage, thereby suffering from lumbago, discomfort of renal region, and the like. To discover a drug that does not jeopardize life-sustaining cells while efficiently killing a specific tumor in a patient will significantly improve the survival rate and quality of life of patients undergoing chemotherapy.

### SUMMARY

The inventors have devoted themselves to developing dipyridyl alkaloid useful in antitumor. The inventors have found that dipyridyl alkaloid is significantly promising in the treatment of tumors.

To this end, the present disclosure provide a compound of formula I, or pharmaceutically acceptable salt or prodrug thereof,

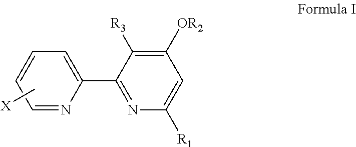

wherein,

R₁ is selected from —CH═N—O—R₄, —COR₅, —CH₂OR₆, —CN, and —NH₂;

R₄ and R₆ are each independently selected from —H, an alkyl group, and an alkanoyl group;

R₅ is selected from —H, —OH, —NH₂, and an alkoxy group;

R₂ is selected from —H, an alkyl group, and a glycosyl group;

R₃ is selected from —H, —OH, —NH₂, and an alkoxy group;

X is selected from —H, halogen, —NO₂, —SO₂R₇, and —COR₈;

R₇ is selected from —H, and an aryl group;

R₈ is an aryl group.

Optionally, provided is the aforementioned compound of formula I, or pharmaceutically acceptable salt or prodrug thereof, wherein,

the alkyl group and alkyl groups from the alkanoyl group and the alkoxy group are each independently a linear or branched C₁ to C₁₆ alkyl group, optionally a linear or branched C₁ to C₁₀ alkyl group, optionally a linear or branched C₁ to C₆ alkyl group, or optionally a linear or branched C₁ to C₄ alkyl group;

the glycosyl group is selected from: glucosyl, rhamnosyl, isorhamnosyl, ribosyl, galactosyl, allosyl, fucosyl, idosyl, talosyl, 2,4-dimethoxyrhamnosyl, 2,4-dimethoxyglucosyl, 2,4-dimethoxyisorhamnosyl, 2,4-dimethoxyribosyl, 2,4-dimethoxygalactosyl, 2,4-dimethoxyallosyl, 2,4-dimethoxyfucosyl, 2,4-dimethoxyidosyl, and 2,4-dimethoxytalosyl;

the halogen is selected from —F, —Cl, —Br, and —I;

the aryl group is a C₆ to C₁₈ monocyclic or polycyclic aryl group, optionally a C₆ to C₁₄ monocyclic or polycyclic aryl group, or optionally a C₆ to C₁₀ monocyclic or polycyclic aryl group.

Optionally, provided is the aforementioned compound of formula I, or pharmaceutically acceptable salt or prodrug thereof, wherein,

R₁ is selected from —CH₂OH, —CH═NOH, —CN, —CONH₂, —NH₂, —CHO, —COOH; optionally, R₁ is —CH═NOH;

R₂ is selected from —H, methyl, glucosyl, rhamnosyl, isorhamnosyl, ribosyl, galactosyl, allosyl, fucosyl, idosyl, talosyl, 2,4-dimethoxyrhamnosyl, 2,4-dimethoxyglucosyl, 2,4-dimethoxyisorhamnosyl, 2,4-dimethoxyribosyl, 2,4-dimethoxygalactosyl, 2,4-dimethoxyallosyl, 2,4-dimethoxyfucosyl, 2,4-dimethoxyidosyl, and 2,4-dimethoxytalosyl; optionally, R₂ is L-rhamnosyl; optionally, R₂ is methyl;

R₃ is selected from —H, —OH, —OCH₃; optionally, R₃ is —H;

X is selected from —H, —F, —Cl, —Br, —I, —NO₂, —SO₃H, —SO₂C₆H₅, —COC₆H₅; optionally, X is —H;

optionally, the compound of formula I is Compound 1, or Compound 2, or Compound 3, or Compound 4, or Compound 5, or Compound 6, or Compound 7:

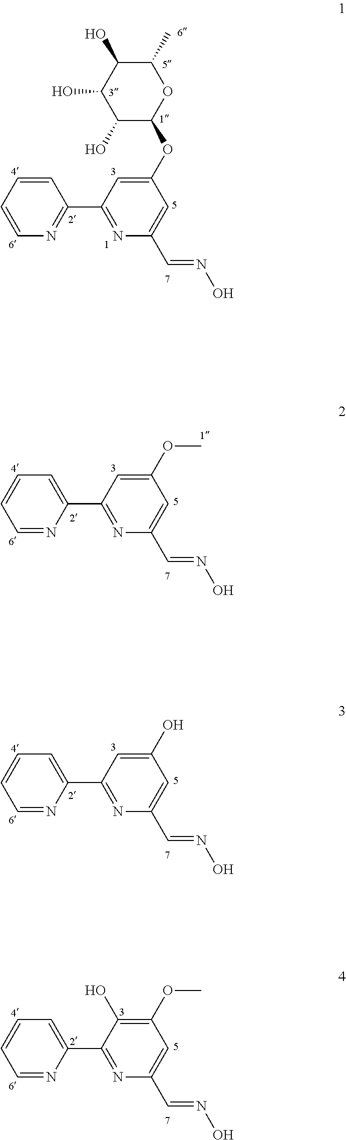 [SMILES: C[C@@H]1O[C@@H](Oc2cc(/C=N/O)nc(-c3ccccn3)c2)[C@H](O)[C@H](O)[C@H]1O ; COc1cc(/C=N/O)nc(-c2ccccn2)c1 ; O/N=C/c1cc(O)cc(-c2ccccn2)n1 ; COc1cc(/C=N/O)nc(-c2ccccn2)c1O]

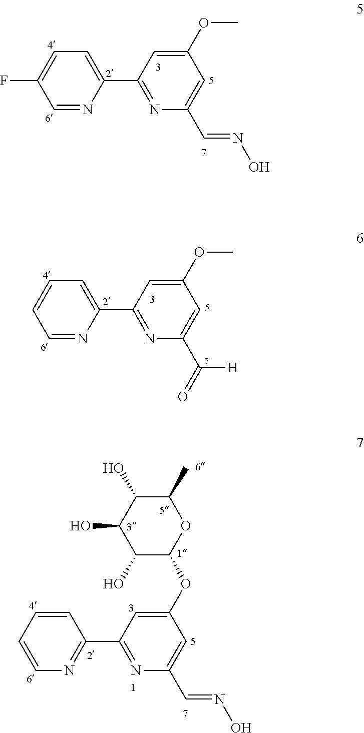 [SMILES: COc1cc(/C=N/O)nc(-c2ccc(F)cn2)c1 ; COc1cc(C=O)nc(-c2ccccn2)c1 ; C[C@H]1O[C@H](Oc2cc(/C=N/O)nc(-c3ccccn3)c2)[C@H](O)[C@@H](O)[C@@H]1O]

Optionally, provided is the aforementioned compound of formula I, pharmaceutically acceptable salt or prodrug thereof, wherein,

the pharmaceutically acceptable salt includes salts of organic or inorganic acids;

optionally, the pharmaceutically acceptable salt is a salt formed by the compound of formula I and a compound selected from the group consisting of: hydrochloric acids, sulfuric acids, phosphoric acids, formic acids, acetic acids, propionic acids, lactic acids, citric acids, tartaric acids, fumaric acids, maleic acids, mandelic acids, malic acids, and camphorsulfonic acids;

the pharmaceutically acceptable prodrug includes prodrugs formed by bonding the compound of formula I to a pharmaceutically acceptable carrier; optionally, the pharmaceutically acceptable carrier includes: triglyceride phosphate, polyethylene glycol ester, polyethylene glycol amide, and polyethylene glycol ether.

The present disclosure further provides a method for preparing the aforementioned compound of formula I, pharmaceutically acceptable salt or prodrug thereof, characterized in comprising: adding a precursor Y to a fermentation medium of A. cyanogriseus WH1-2216-6, subjecting the A. cyanogriseus WH1-2216-6 to fermentation cultivation, separating and purifying the fermented product to give a product; optionally, the product is prepared by subjecting a isolated and purified product to semi-synthesis,

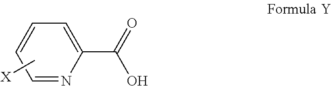

wherein X is as defined above;

optionally, the fermentation cultivation comprises: culturing A. cyanogriseus WH1-2216-6 in a seed medium, inoculating the culture to a fermentation medium, culturing and fermenting it to give a fermented product;

optionally, the seed medium comprises: a carbon source, a nitrogen source, and a sodium chloride-containing aqueous solution; optionally, the seed culture medium comprises: peptone, glycerin, soybean flour, soluble starch, calcium carbonate, and a sodium chloride-containing aqueous solution (preferably natural seawater); optionally, the seed medium is consisting of: 15 parts by weight of peptone, 15 parts by weight of glycerin, 5 parts by weight of soybean flour, 15 parts by weight of soluble starch, 2 parts by weight of CaCO₃, and 1000 parts by weight of aged seawater, pH=7.8;

optionally, the fermentation medium comprises: soluble starch, glycerin, peptone, calcium carbonate, macroporous resin, seawater; optionally, the fermentation medium is consisting of: 20 parts by weight of soluble starch, 20 parts by weight of glycerin, 20 parts by weight of peptone, 2 parts by weight of CaCO₃, XAD-16 macroporous resin, 1000 parts by weight of aged seawater, pH=7.5;

optionally, the isolation and purification comprise: extracting the fermented product with an organic solvent, after concentration, the organic phase being added an acidic solution, followed by extraction with an organic solvent; removing the organic phase; adding an alkali to the aqueous phase to adjust the pH; extracting with an organic solvent; concentrating the organic phase to obtain an alkaloid fraction; and separating the resulting alkaloid fraction by methods selected from the group consisting of gel column chromatography, silica gel column chromatography (optionally, vacuum silica gel column chromatography, reversed-phase vacuum silica gel column chromatography, and silica gel column flash chromatography), and semi-preparative high performance liquid chromatography; optionally, organic solvents for each extraction are independently selected from ethyl acetate, dichloromethane, chloroform, and petroleum ether; optionally, the acidic solution is selected from hydrochloric acid, sulfuric acid, nitric acid, and phosphoric acid; optionally, the base is selected from ammonium hydroxide, sodium hydroxide, potassium hydroxide, sodium carbonate, and potassium carbonate; optionally, the eluant for the gel column chromatography is selected from methanol, and a dichloromethane-methanol mixture; optionally, the eluant for the silica gel column chromatography is selected from petroleum ether, dichloromethane, methanol, dichloromethane-methanol mixture, and water; optionally, the eluant for the reversed-phase vacuum silica gel column chromatography is selected from methanol and water; optionally, the eluant for the semi-preparative high performance liquid chromatography is selected from a methanol-water mixture, and an acetonitrile-water mixture; optionally, the eluant for the vacuum silica gel column chromatography is selected from petroleum ether, dichloromethane, and a dichloromethane-methanol mixture; optionally, the eluant for the Sephadex LH-20 gel column chromatography is methanol, or a dichloromethane-methanol mixture; optionally, the eluant for the silica gel column flash chromatography is dichloromethane, or methanol; optionally, the eluant for the reversed-phase vacuum gel column chromatography is selected from methanol and water; optionally, the eluant for the semi-preparative high performance liquid chromatography is selected from a methanol-water mixture, and an acetonitrile-water mixture;

optionally, the semi-synthesis reaction using the isolated and purified product as raw material comprises:

(i) when R₁ is selected from —CH═N—O—R₄, —COR₅, —CH₂OR₆, —CN, and —NH₂, a isolated compound, in which R₁ is —CH═N—OH or —CH₂OH or —CONH₂, as a starting material is undergone a chemical reaction with a common chemical reagent, or with halohydrocarbon R₄Cl, or formaldehyde, or chromic acid, or halohydrocarbon R₆Cl, or acetic anhydride, or potassium hydroxide, and thereby a compound containing these groups is obtained through an alkylation reaction, or an oxidation reaction, or a reduction reaction, or a hydrolysis reaction, respectively;

(ii) when R₂ is an alkyl group or a glycosyl group, a isolated compound, in which R₂ is —H, as a starting material is undergone a chemical reaction with common halohydrocarbon R₂Cl, or sugar, and thereby a compound containing these groups is obtained through an alkylation reaction, or a glycosylatoin reaction, respectively;

(iii) when R₃ is —NH₂ or an alkoxyl group, a isolated compound, in which R₃ is —OH, as a starting material is undergone a chemical reaction with a common haloalkane, and —NH₂ and an alkoxyl group in a benzene ring are introduced through a halogenation reaction, a replacement reaction, or an alkylation reaction.

The above reactions may be carried out by known methods in the art.

Optionally, the compound of formula I is Compound 1, or Compound 2, or Compound 3, or Compound 4, or Compound 5, or Compound 6, or Compound 7;

optionally, Compounds 1 to 4 are obtained according to the following methods:

(a) the A. cyanogriseus WH1-2216-6 is cultured in a seed medium, inoculated to a fermentation medium, to which 2-picolinic acid is added, and subjected to submerged cultivation and fermentation to obtain a fermented product; the fermented product is extracted with an organic solvent, and the organic phase is concentrated to obtain a crude extract, to which a HCl solution is added; the crude extract is extracted with an organic solvent, and the aqueous phase is adjusted to pH=8.0 with ammonium hydroxide, followed by extraction with an organic solvent, and the organic phase is concentrated to obtain an alkaloid fraction; the resulting alkaloid fraction is separated over a gel column having a mobile phase of dichloromethane:methanol=1:1 (v/v), to obtain Compound 2 and other alkaloid fractions;

(b) the other alkaloid fractions obtained in step (a) are combined and separated through silica gel column chromatography under reduced pressure, by gradiently eluting with petroleum ether, dichloromethane, and dichloromethane-methanol as eluants in this order;

wherein the dichloromethane-methanol eluant includes components at ratios of: dichloromethane:methanol (v/v) being 100:1, 50:1, 30:1, 25:1, 15:1, 10:1, 8:1, 5:1, 2:1, 1:1, and 0:1;

wherein the obtained fractions from the elution by the eluant of dichloromethane and the elution by the eluant of dichloromethane:methanol=100:1 (v/v) out of the dichloromethane-methanol eluants are combined and chromatographed over a gel (Sephadex LH-20) column by eluting with dichloromethane:methanol=1:1 (v/v), and the resulting component is chromatographed over a gel (Sephadex LH-20) column by eluting with methanol to give Compound 4;

wherein the obtained fractions from the elution by the eluant of dichloromethane:methanol=8:1 (v/v) out of the dichloromethane-methanol eluants is successively undergone gel (Sephadex LH-20) column chromatography by eluting with methanol, and undergone silica gel column flash chromatography by a gradient elution with dichloromethane and methanol to obtain 5 fractions, as Fraction 1, Fraction 2, Fraction 3, Fraction 4, and Fraction 5 in the elution order; Fraction 2 is purified by semi-preparative high performance liquid chromatography (HPLC) by eluting with methanol:water=50:50 (v/v) to obtain Compound 1; Fraction 3 is purified by semi-preparative high performance liquid chromatography by eluting with methanol:water=30:70 (v/v) to obtain Compound 3;

optionally, Compound 5 is obtained according to the following method: the A. cyanogriseus WH1-2216-6 is cultured in a seed medium, inoculated into a fermentation medium, to which 5-fluoropicolinic acid (precursor 9) is added, and subjected to submerged cultivation and fermentation to obtain a fermented product; the fermented product is extracted with an organic solvent, and the organic phase is concentrated to obtain a crude extract, the crude extract is separated through silica gel column chromatography under reduced pressure by gradiently eluting with petroleum ether, dichloromethane, and dichloromethane-methanol mixture as eluants in this order; the resulting fraction is successively eluted with methanol over a gel (Sephadex LH-20) column, undergone silica gel column flash chromatography and subjected to gradient elution with dichloromethane and methanol; after concentration, the resulting fracgtion is purified by semi-preparative high performance liquid chromatography by eluting with methanol:water=75:25 (v/v) to obtain Compound 5;

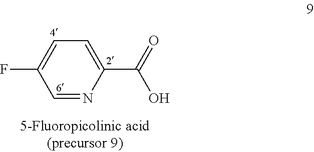

optionally, Compound 6 is obtained by the following synthesis method: formalin, hydrochloric acid and water are added to the Compound 2 obtained by separation, the mixture is refluxed, and then cooled to room temperature, to which a saturated aqueous solution of NaHCO₃ is added; the mixture is extracted with an organic solvent; after vacuum concentration, the organic phase is separated over a silica gel column under increased pressure by eluting with an eluant selected from ethyl acetate, dichloromethane, chloroform, and petroleum ether, and preferably, gradient elution is conducted with dichloromethane and methanol as eluants, to obtain Compound 6; optionally, the concentration of the formalin is 37% (v/v); optionally, the concentration of the hydrochloric acid is 10N;

Compound 7 is obtained by the following method: the A. cyanogriseus WH1-2216-6 was cultured in a seed medium, inoculated into a fermentation medium, to which 2-picolinic acid was added, subjected to submerged cultivation and fermentation to obtain a fermented product; the fermented product is extracted with an organic solvent, and the organic phase is concentrated to obtain a crude extract, the crude extract is separated through silica gel column chromatography under reduced pressure by gradiently eluting with petroleum ether, dichloromethane, and dichloromethane-methanol mixed solvent (v/v, 100:1, 50:1, 30:1, 25:1, 15:1, 10:1, 5:1, 2:1, 1:1, 0:1) as eluants in this order; the obtained fraction from the elution by the eluant of dichloromethane-methanol=2:1 (v/v) out of the dichloromethane-methanol eluants is purified through semi-preparative high performance liquid chromatography by eluting with methanol:water=50:50 (v/v) to obtain Compound 7;

optionally, the organic solvent for extraction is selected from ethyl acetate, dichloromethane, chloroform, and petroleum ether.

The present disclosure also provides a pharmaceutical composition, wherein the pharmaceutical composition comprises at least one selected from the compound of formula I, pharmaceutically acceptable salt or prodrug thereof, and a pharmaceutically acceptable excipient.

Optionally, the pharmaceutical composition described above is characterized in that the dosage form of the pharmaceutical composition includes: a solid preparation and a liquid preparation; optionally, the dosage form of the pharmaceutical composition includes an oral preparation, an injection preparation, a transdermal preparation; optionally, the dosage form of the pharmaceutical composition includes a tablet, a capsule, a powder, a granule, a lozenge, a suppository, an oral solution, a sterile parenteral suspension, an injection; optionally, the injection includes a frozen-dried powder injection.

Optionally, provided is use of the above compound of formula I, or pharmaceutically acceptable salt or prodrug thereof, or use of the above pharmaceutical composition in the preparation of an antitumor drug; optionally, the antitumor drug is a tumor cell proliferation inhibitor or a tumor cell killer; optionally, the antitumor drug is devoid of drugs against human lung adenocarcinoma A549, drugs against human acute promyelocytic leukemia HL60, drugs against human chronic myeloid leukemia K562, and drugs against human oral epidermoid carcinoma KB; optionally, the antitumor drug is a drug against human colon cancer cell line HCT-116, a drug against human breast cancer cell line MCF-7, a drug against hepatoma cell line HepG2, a drug against cervical cancer cell line Hela, or a drug against human peripheral blood leukemia T cell line Jurkat.

The present disclosure further provides use of the above compound of formula I, or pharmaceutically acceptable salt or prodrug thereof as a probe for inhibiting cell proliferation.

The present disclosure further provides use of the above pharmaceutical composition as a probe for inhibiting cell proliferation.

The present disclosure further provides a probe kit, characterized in comprising the above compound of formula I, or pharmaceutically acceptable salt or prodrug thereof.

The present disclosure further provides a probe kit, characterized in comprising the above pharmaceutical composition.

Optionally, the probe kit further comprises a biocompatible medium; optionally, the biocompatible medium is at least one selected from methanol, water, and dimethyl sulfoxide.

The A. cyanogriseus WH1-2216-6 used for the preparation of the compound of formula I of the present disclosure was deposited on Nov. 28, 2009 with the accession number of the deposit of CCTCC M 209277, at the China Center for Type Culture Collection at the Wuhan University, in Wuhan Province, China

The actinomycete strain WH1-2216-6 was isolated from sea mud collected from Weihai, Shandong Province, and the detailed information on the strain has been reported (see Fu P, Wang S, Hong K, Li X, Liu P, Wang Y, Zhu W. Cytotoxic bipyridines from the marine-derived actinomycete Actinoalloteichus cyanogriseus WH1-2216-6. J. Nat. Prod. 2011, 74, 1751-1756), which was identified as an Actinoalloteichus cyanogriseus strain and designated Actinoalloteichus cyanogriseus WH1-2216-6 after polyphasic taxonomic studies. In the method of preparing the compound of the present disclosure by fermenting microorganisms, any microorganisms capable of producing the compound of the present disclosure may be used, as long as the microorganisms are capable of producing the compound of the present disclosure can all be used as producing strains for the preparation of the compound of the present disclosure.

The compound of the present disclosure has good antitumor activity and good selectivity, and has a significant inhibitory effect on tumor cell proliferation, while having a non-obvious inhibitory effect on growth of normal cell lines.

### BRIEF DESCRIPTION OF THE DRAWINGS

FIG. 1 is a picture of colon cancer (HCT116) of mice from respective groups in Test Example 2, wherein Group A is a negative control (normal saline) group, Group B is Docetaxel (5 mg/kg) group, and Group C is Compound 2 of the present disclosure (7.5 mg/kg) group.

### DETAILED EMBODIMENTS

Examples are provided below to illustrate the present disclosure, but the scope of the present invention is not limited to these examples.

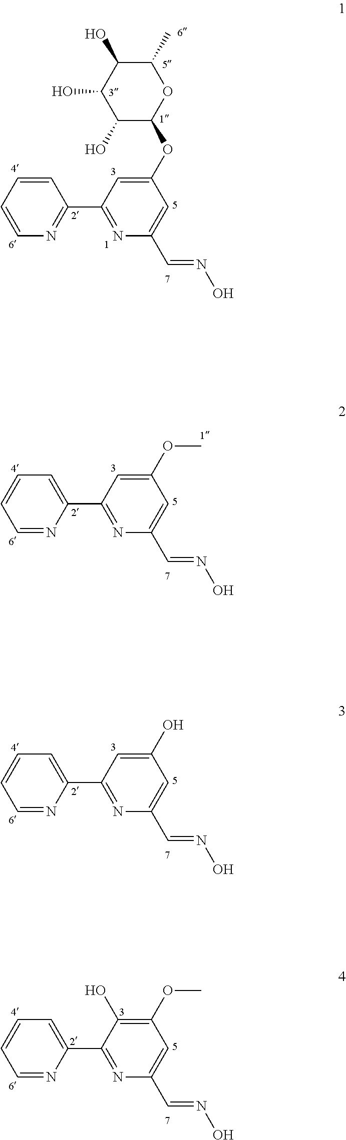 [SMILES: C[C@@H]1O[C@@H](Oc2cc(/C=N/O)nc(-c3ccccn3)c2)[C@H](O)[C@H](O)[C@H]1O ; COc1cc(/C=N/O)nc(-c2ccccn2)c1 ; O/N=C/c1cc(O)cc(-c2ccccn2)n1 ; COc1cc(/C=N/O)nc(-c2ccccn2)c1O]

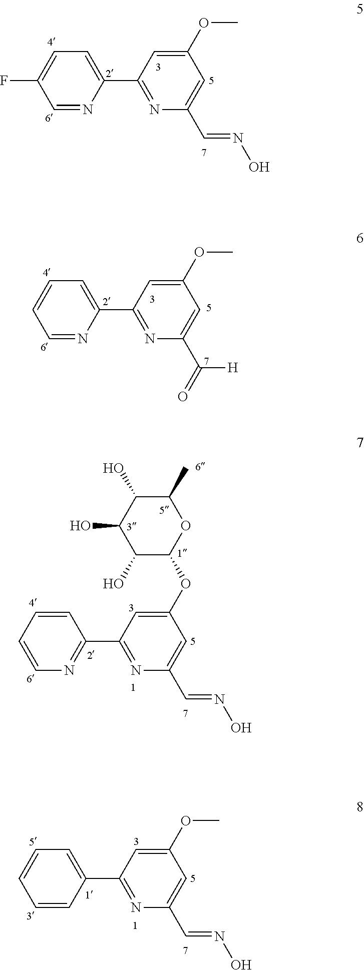 [SMILES: COc1cc(/C=N/O)nc(-c2ccc(F)cn2)c1 ; COc1cc(C=O)nc(-c2ccccn2)c1 ; C[C@H]1O[C@H](Oc2cc(/C=N/O)nc(-c3ccccn3)c2)[C@H](O)[C@@H](O)[C@@H]1O ; COc1cc(/C=N/O)nc(-c2ccccc2)c1]

### PREPARATION EXAMPLES

(1) Preparations of Compounds 1 to 4, 7, and 8

Fermentation Cultivation: The A. cyanogriseus WH1-2216-6 was cultured for 5 days in a seed medium, and inoculated into a 500 mL flask filled with 150 mL of fermentation medium, to which 0.4 g of 2-picolinic acid was added. The culture was subjected to submerged cultivation and fermented for 12 days to obtain 40 L of a fermented product. The medium comprised: a seed medium (15 g of peptone, 15 g of glycerol, 5 g of soybean flour, 15 g of soluble starch, 2 g of CaCO₃, and 1 L of aged seawater, pH=7.8); and a fermentation medium (20 g of soluble starch, 20 g of glycerin, 20 g of peptone, 2 g of CaCO₃, 50 g of XAD-16 macroporous resin, and 1 L of aged seawater, pH=7.5).

Isolation and Refinement

(a) Production of Compound 2: The fermented product was extracted trice with isometric ethyl acetates, and the organic phases of the ethyl acetate extracts were combined and concentrated to give a crude extract. To every gram of the crude extract was added 50 mL of 3% HCl solution, stirred, allowed to stand, and extracted thrice with isometric dichloromethanes, and the aqueous phase was adjusted to pH=8.0 with ammonium hydroxide and then extracted with dichloromethane. The organic phase was concentrated to give 35.0 g of alkaloid fraction. The alkaloid fraction was separated twice over a gel column (mobile phase: dichloromethane:methanol=1:1, v/v) to obtain Compound 2 and other alkaloid fractions. With HPLC analysis, the purity of Compound 2 was 99% or more.

(b) Production of Compounds 1, 3 and 4: After combined, the other alkaloid fractions obtained from the above step (a) were chromatographed over a silica gel column under reduced pressure by gradiently eluting with petroleum ether, dichloromethane, and dichloromethane-methanol mixed solvent (v/v, 100:1, 50:1, 30:1, 25:1, 15:1, 10:1, 8:1, 5:1, 2:1, 1:1, 0:1) as eluants in this order:

wherein the obtained fractions from the elution by the eluant of dichloromethane and the elution by the eluant of the dichloromethane:methanol=100:1 (v/v) out of the dichloromethane-methanol eluants were combined and separated over a Sephadex LH-20 column by eluting with dichloromethane:methanol=1:1 (v/v), and the resulting fraction was chromatographed over a Sephadex LH-20 column by eluting with methanol to give a Compound 4 (70.0 mg);

wherein the obtained fractions from the elution by the eluant of dichloromethane:methanol=8:1 (v/v) out of the dichloromethane-methanol eluants was undergone Sephadex LH-20 column chromatography (eluting with methanol), and silica gel column flash chromatography (gradiently eluting with dichloromethane and methanol) to obtain 5 fractions as Fraction 1, Fraction 2, Fraction 3, Fraction 4, and Fraction 5 in the elution order. Fraction 2 was purified by semi-preparative high performance liquid chromatography by eluting with methanol:water=50:50 (v/v) to obtain Compound 1 (4.5 mg, retention time: t_{R}=7.5 min). Fraction 3 was purified by semi-preparative high performance liquid chromatography by eluting with methanol:water=30:70 (v/v) to obtain Compound 3 (3.0 mg, retention time: t_{R}=13.7 min).

Compound 1: lavender powder; high resolution mass spectrum HRESIMS m/z 362.1343 [M+H]⁺ (calcd for C₁₇H₂₀N₃O₆, 362.1347); optical rotation [α]_{D}¹⁵ −50.6 (c 0.1 CH₃OH); ultraviolet absorption spectrum UV (CH₃OH) λₘₐₓ (log ε) 212 (4.33), 240 (4.17), 285 (3.91) nm; infrared spectrum IR (KBr) νₘₐₓ 3547, 3327, 3325, 2929, 2376, 2304, 1679, 1586, 1564, 1515, 1455, 1427, 1383, 1203, 1136, 1024, 839, 798, 720 cm⁻¹; data about nuclear magnetism ¹H NMR spectrum (500 MHz, DMSO-d₆) and ¹³C NMR spectrum (125 MHz, DMSO-d₆) were listed in Table 1.

Compound 2: white powder; low resolution mass spectrum ESIMS m/z 230.2 [M+H]⁺; data about nuclear magnetism ¹H NMR spectrum (500 MHz, DMSO-d₆) and ¹³C NMR spectrum (125 MHz, DMSO-d₆) were listed in Table 1.

Compound 3: white powder; high resolution mass spectrum HRESIMS m/z 216.0781 [M+H]⁺ (calcd for C₁₁H₁₀N₃O₂, 216.0773); UV (MeOH) λₘₐₓ (log ε): 240 (2.91), 280 (2.75) nm; infrared spectrum IR (KBr) νₘₐₓ 3171, 2953, 1651, 1502, 1378, 1302, 1016, 980, 797, 738 cm⁻¹; data about nuclear magnetism ¹H NMR spectrum (600 MHz, DMSO-d₆) and ¹³C NMR spectrum (150 MHz, DMSO-d₆) were listed in Table 2.

Compound 4: yellow powder; high resolution mass spectrum ESIMS m/z 246.096 [M+H]⁺; optical rotation [α]_{D}¹⁵ −5.2 (c 0.2, MeOH); ultraviolet absorption spectrum UV (MeOH) λₘₐₓ (log ε) 245 (4.22), 324 (3.80) nm; infrared spectrum IR (KBr) νₘₐₓ 3563, 3445, 2927, 1646, 1566, 1540, 1515, 1380, 1261, 1151, 1050, 991, 865, 800 cm⁻¹; data about nuclear magnetism ¹H NMR spectrum (500 MHz, DMSO-d₆) and ¹³C NMR spectrum (125 MHz, DMSO-d₆) were listed in Table 2.

TABLE 1
Data about ¹H and ¹³C NMR of Compounds 1 and 2

| | 1 | | 2 | |
|---|---|---|---|---|
| Positions | δ_{C} | δ_{H} (J in Hz) | δ_{C} | δ_{H} (J in Hz) |
| 2 | 157.0, C | | 156.7, C | |
| 3 | 108.3, CH | 8.00, d (1.9) | 106.5, CH | 7.91, d (2.5) |
| 4 | 163.7, C | | 166.6, C | |
| 4-OCH₃ | | | 55.6, CH₃ | 3.95, s |
| 5 | 107.2, CH | 7.45, d (2.0) | 105.6, CH | 7.33, d (2.4) |
| 6 | 153.6, C | | 153.4, C | |
| 7 | 148.7, CH | 8.15, s | 148.7, CH | 8.15, s |
| 7-NOH | | 11.77, s | | 11.74, s |
| 2′ | 154.4, C | | 154.4, C | |
| 3′ | 120.7, CH | 8.38, d (8.0) | 120.8, CH | 8.39, d (8.0) |
| 4′ | 137.4, CH | 7.96, t (7.8) | 137.5, CH | 7.97, td (7.7, 1.8) |
| 5′ | 124.6, CH | 7.48, t (6.0) | 124.6, CH | 7.48, dd (6.4, 4.8) |
| 6′ | 149.3, CH | 8.69, d (4.1) | 149.2, CH | 8.69, d (3.2) |
| 1″ | 98.1, CH | 5.63, s | | |
| 2″ | 69.8, CH | 3.88, s | | |
| 2″-OH | | 5.17, d (4.2) | | |
| 3″ | 70.1, CH | 3.67, m | | |
| 3″-OH | | 4.83, d (5.9) | | |
| 4″ | 71.6, CH | 3.29, m | | |
| 4″-OH | | 4.92, d (5.8) | | |
| 5″ | 70.3, CH | 3.43, in | | |
| 6″ | 17.9, CH₃ | 1.11, d (6.1) | | |

TABLE 2
Data about ¹H and ¹³C NMR of Compounds 3 and 4

| | 3 | | 4 | |
|---|---|---|---|---|
| Positions | δ_{C} | δ_{H} (J in Hz) | δ_{C} | δ_{H} (J in Hz) |
| 2 | 157.8, C | | 134.1, C | |
| 3 | 108.0, CH | 7.80, d (2.3) | 147.2, C | |
| 3-OH | | | | 14.63, s |
| 4 | 166.1, C | | 155.2, C | |
| 4-OH | | 10.95, brs | 55.7, CH₃ | 3.92, s |
| 5 | 109.4, CH | 7.21, d (2.3) | 103.5, CH | 7.38, s |
| 6 | 154.3, C | | 143.4, C | |
| 7 | 150.0, CH | 8.10, s | 148.8, CH | 8.05, s |
| 7-NOH | | 11.63, s | | 11.52, s |
| 2′ | 155.8, C | | 157.1, C | |
| 3′ | 121.7, CH | 8.35, d (7.6) | 120.6, CH | 8.52, d (8.1) |
| 4′ | 138.2, CH | 7.92, t (6.5) | 138.9, CH | 8.11, t (7.7) |
| 5′ | 125.4, CH | 7.45, t (6.5) | 123.7, CH | 7.55, t (6.3) |
| 6′ | 150.3, CH | 8.66, d (4.5) | 145.8, CH | 8.66, d (4.4) |

(2) Preparation of Compound 5

Fermentation Cultivation (having 5-fluoropicolinic acid as a precursor added): The A. cyanogriseus WH1-2216-6 was cultured for 5 days in a seed medium, and inoculated into 150 mL of fermentation medium, to which 0.4 g of 5-fluoropicolinic acid as a precursor was added. The culture was subjected to submerged cultivation and fermented for 12 days into 7 bottles in total to obtain 1.0 L of a fermented product. The composition of the medium was as follows: a seed medium (15 g of peptone, 15 g of glycerol, 5 g of soybean flour, 15 g of soluble starch, 2 g of CaCO₃, and 1 L of aged seawater, pH=7.8); and a fermentation medium (20 g of soluble starch, 20 g of glycerin, 20 g of peptone, 2 g of CaCO₃, 50 g of XAD-16 macroporous resin, and 1 L of aged seawater, pH=7.5).

Isolation and Refinement: The fermented product was extracted trice with isometric ethyl acetates, and the organic phases were combined and concentrated to give a crude extract. The resulting crude extract was chromatographed over a silica gel column under reduced pressure by gradiently eluting with petroleum ether, dichloromethane, and dichloromethane-methanol as eluants in this order. After concentration, the fraction was undergone Sephadex LH-20 column chromatography (eluting with methanol), and silica gel column flash chromatography (gradiently eluting with dichloromethane and methanol) successively. The resulting fraction was separated through semi-preparative high performance liquid chromatography by eluting with methanol:water=75:25 (v/v) to obtain Compound 5 (10.0 mg, retention time: t_{R}=10.3 min).

Compound 5: white crystalline powder; high resolution mass spectrum HRESIMS m/z 248.0831 [M+H]⁺ (calcd for C₁₂H₁₁N₃O₂F, 248.0830); ultraviolet absorption spectrum UV (MeOH) λₘₐₓ (log ε) 242 (4.69), 280 (4.30) nm; infrared spectrum IR (KBr) νₘₐₓ 3744, 3674, 3227, 3110, 2924, 1738, 1650, 1583, 1489, 1464, 1359, 1239, 1166, 1112, 1026, 982, 930, 840, 767, 714 cm⁻¹; data about nuclear magnetism ¹H NMR spectrum (500 MHz, DMSO-d₆) and ¹³C NMR spectrum (125 MHz, DMSO-d₆) were listed in Table 3.

(3) Preparation of Compound 6

Reaction Steps: Compound 2 (30.0 mg, 0.13 mmol) was placed in a 25 mL reaction flask, to which 2.28 mL of 37% formalin, 0.116 mL of 10N hydrochloric acid and 1.032 mL of water were added, followed by refluxing at 105° C. for 30 minutes. The reaction mixture was cooled to room temperature and then 10 mL of saturated aqueous NaHCO₃ was added to quench the reaction.

Isolation and Refinement: The reaction mixture was extracted trice with isometric ethyl acetates (10 mL/time). The organic layer of the ethyl acetate was subjected to vacuum concentration, purified through a silica gel column under increased pressure by gradiently eluting with dichloromethane and methanol to give Compound 6 (25.0 mg, in a yield of 89%).

Compound 6: colorless acicular crystal; high resolution mass spectrum HRESIMS m/z 215.0817 [M+H]⁺ (calcd for C₁₂H₁₁N₂O₂ₐ, 215.0815); ultraviolet absorption spectrum UV (MeOH) λₘₐₓ (log ε): 215 (4.43) nm; infrared spectrum IR (KBr) νₘₐₓ 3407, 3089, 2919, 2815, 1715, 1584, 1374, 1220, 1046 cm⁻¹; data about nuclear magnetism ¹H NMR spectrum (600 MHz, CDCl₃) and ¹³C NMR spectrum (150 MHz, CDCl₃) were listed in Table 3.

TABLE 3
Data about ¹H and ¹³C NMR of Compounds 5 and 6

| | 5 | | 6 | |
|---|---|---|---|---|
| Positions | δ_{C} | δ_{H} (J in Hz) | δ_{C} | δ_{H} (J in Hz) |
| 2 | 155.9, C | | 155.1, C | |
| 3 | 106.4, CH | 7.83, d (2.2) | 107.8, CH | 7.48, d (2.5) |
| 4 | 166.7, C | | 167.7, C | |
| 4-OCH₃ | 55.6, CH₃ | 3.94, s | 56.0, CH3 | 4.00, s |
| 5 | 105.5, CH | 7.32, d (2.2) | 110.6, CH | 8.17, d (2.5) |
| 6 | 153.5, C | | 158.5, C | |
| 7 | 148.7, CH | 8.14, s | 193.8, CH | 10.12, s |
| 7-NOH | | 11.77, s | | |
| 2′ | 151.2 (d, ⁴J_{CF} = 3 Hz), C | | 154.2, C | |
| 3′ | 122.4 (d, ³J_{CF} = 5 Hz), CH | 8.44, dd (8.8,4.7) | 121.6, CH | 8.53, d (7.9) |
| 4′ | 124.2 (d, ²J_{CF} = 19 Hz), CH | 7.88, td (8.7, 2.8) | 137.2, CH | 7.86, td (7.8, 1.8) |
| 5′ | 159.7 (d, ¹J_{CF} = 256 Hz), C | | 124.5, CH | 7.36, dd (7.4, 4.7) |
| 6′ | 137.3 (d, ²J_{CF} = 24 Hz), CH | 8.68, d (2.6) | 149.3, CH | 8.69, d (4.7) |

(4) Preparation of Compounds 7 and 8

Compound 7 was obtained from the method below: The A. cyanogriseus WH1-2216-6 was cultured in a seed medium, and inoculated into a fermentation medium, to which 2-picolinic acid was added. The culture was subjected to submerged cultivation and fermented to obtain a fermented product. The fermented product was extracted with an organic solvent, and the organic phase was concentrated to obtain a crude extract, the crude extract was separated through silica gel column chromatography under reduced pressure by gradiently eluting with petroleum ether, dichloromethane, and dichloromethane-methanol mixed solvent (v/v, 100:1, 50:1, 30:1, 25:1, 15:1, 10:1, 5:1, 2:1, 1:1, 0:1) as eluants in this order, the obtained fraction from the elution by the eluant of dichloromethane:methanol=2:1 (v/v) out of the dichloromethane-methanol eluants was purified through semi-preparative high performance liquid chromatography by eluting with methanol:water=50:50 (v/v) to obtain Compound 7 (2.3 mg, retention time: t_{R}=12.1 min).

Preparation of Compound 8: The A. cyanogriseus WH1-2216-6 was cultured in a seed medium, and inoculated to a fermentation medium, to which benzoic acid was added. The culture was subjected to submerged cultivation and fermented to obtain a fermented product. The fermented product was extracted with an organic solvent, and the organic phase was concentrated to obtain a crude extract, the crude extract is separated through silica gel column chromatography under reduced pressure by gradiently eluting with petroleum ether, dichloromethane, and dichloromethane-methanol mixed solvent (v/v, 100:1, 50:1, 30:1, 25:1, 15:1, 10:1, 5:1, 2:1, 1:1, 0:1) as eluants in this order, the obtained fraction from the elution by the eluant of dichloromethane:methanol=30:1 (v/v) out of the dichloromethane-methanol eluant was chromatographed over Sephadex LH-20 column by eluting with dichloromethane:methanol=1:1 (v/v). The resulting fraction was then purified by semi-preparative high performance liquid chromatography by eluting with methanol:water=60:40 (v/v) to obtain Compound 8 (3.5 mg, retention time: t_{R}=13.3 min).

Structures of Compounds 7 and 8 were identified in comparison with standards through thin-layer chromatography (TLC) and HPLC (Compound 7: Fu P, Zhu Y, Mei X, Wang Y, Jia H, Zhang C, Zhu W. Acyclic Congeners from Actinoalloteichus cyanogriseus Provide Insights into Cyclic Bipyridine Glycoside Formation. Org. Lett. 2014, 16, 4264-4267; Compound 8: Fu P, Wang S, Hong K, Li X, Liu P, Wang Y, Zhu W. Cytotoxic bipyridines from the marine-derived actinomycete Actinoalloteichus cyanogriseus WH1-2216-6. J. Nat. Prod. 2011, 74, 1751-1756).

### [Test Example 1] Test for In Vitro Antitumor Activity

1. Experimental Samples and Experimental Methods

Formulation of sample solutions under test: Test samples were Compounds 1 to 8 prepared by isolation in the above Preparation Example 1. Appropriate amounts of Compounds 1 to 8 were accurately weighed and solutions of the desired concentrations were formulated with methanol for the activity test.

Cell lines and subculture of cells: The following five tumor cell lines were used for activity test human colon cancer cell lines (HCT-116 cells), human hepatoma cell lines (HepG2 cells), human breast cancer cell lines (MCF-7 cells), cervical cancer cell lines (Hela cells), human peripheral blood leukemia T cell lines (Jurkat cells), and human normal liver cell lines (L-02 cells), of which MCF-7, HepG2, Hela, and L-02 cell lines were tested using the MTT model; HCT-116 cell lines were tested using the SRB model. All of the cells were subcultured in a 10% FBS-containing RPMI-1640 medium in an incubator charged with 5% carbon dioxide at 37° C.

MTT method: Dehydrogenase in mitochondria in live cells was capable of metabolizing yellow bromized 3-(4,5-dimethylthiazole)-2,5-diphenyltetrazolium into ianthinus water-insoluble formazan, and an amount of formazan could be determined by measuring the absorbance thereof with a microplate reader. Since the amount of formazan was directly proportional to the number of live cells, the number of live cells could be determined from the absorbance to learn about the drug's capability of inhibiting or killing tumor cells. During the activity test, the cell lines at the logarithmic phase were prepared by using fresh RPMI-1640 medium into a cell suspension with a density of 3×10⁴ cells/mL. The cell suspension was inoculated to a 96-well plate at 100 μL per well, and cultured at 37° C. for 24 hours. Thereafter, 100 μL of the sample solutions at different concentrations were added to respective wells and continued to be cultured for 72 hours. Then, 20 μL of MTT-containing IPMI-1640 solution (5 mg/L) was added. After further cultivation for 4 hours, the culture solutions were slowly decanted and 150 μL of DMSO was added to dissolve the formazan, and the absorbance was measured at a wavelength of 540 nm. The cell proliferation inhibition rate (IR %) at each concentration was calculated according to the following equation: IR %=(OD_{blank control}−ODₛₐₘₚₗₑ)/OD_{blank control}×100%. The half inhibitory concentration against cancer cells (IC₅₀) and the half inhibitory concentration against normal cells (CC₅₀) were determined.

SRB method: According to the cell growth rate, tumor cells at the logarithmic phase (medium: RPMI-1640 medium containing 10% newborn fetal bovine serum (FBS); cell density: 3×10⁴ cells/mL) were inoculated into a 96-well culture plate at 180 μL/well, and subjected to adherent growth for 24 hours at 37° C. under 5% CO₂. Subsequently, a test sample was added at 20 μL/well, and four duplicated wells were provided at each concentration. (The final concentration of the sample at the preliminary screening was set to 10 μM, and 5 to 7 concentration gradients were set by a double dilution method at the time of testing IC₅₀; the positive drug is 1 μM of doxorubicin; the blank control is addition of the isometric medium at the corresponding concentration.) The tumor cells after dosing were further cultured for 72 hours at 37° C. under 5% CO₂. The culture solution was decanted, and the cells were fixed with 10% cold trichloroacetic acid (TCA), allowed to stand at 4° C. for 1 hour, and then washed 5 times with distilled water and dried in the air. Thereafter, 100 μL/well of a 4 mg/ml solution of sulforhodamine B (SRB, Sigma) formulated by 1% glacial acetic acid was added, and the mixture was stained at a room temperature for 15 minutes, and the supernatant was removed, washed 5 times with 1% acetic acid, and dried in the air. Finally, 150 μL/well of Tris solution was added, and the absorbance (OD value) was measured with a microplate reader at a wavelength of 540 nm. The cell proliferation inhibition rate (IR %) at each concentration was calculated according to the equation: IR %=(OD_{blank control}−ODₛₐₘₚₗₑ)/OD_{blank control}×100%. The half inhibitory concentration against cancer cells (IC₅₀) was determined.

Selection Index (SI) is the ratio of the CC₅₀ value of a test sample inhibiting the growth of normal cell lines to the IC₅₀ value of the test sample inhibiting tumor cell proliferation, which reflects the selectivity and safety of the sample.

SI=CC₅₀(normal cell line L-02)/IC₅₀(tumor cell line) Equation:

2. Experimental Results

TABLE 4
Toxicity CC₅₀ (μM) of Respective Compounds to Normal Cells

| Cell | | | | | | | | |
|---|---|---|---|---|---|---|---|---|
| Line | Compound 1 | Compound 2 | Compound 3 | Compound 4 | Compound 5 | Compound 6 | Compound 7 | Compound 8 |
| L-02 | 28.3 | 3.9 | 2.5 | 1.7 | 7.5 | 31.4 | 59.5 | >50 |
| (CC₅₀) | | | | | | | | |

TABLE 5
Cytotoxic Activity IC₅₀ (μM) and SI of Respective Compounds to HCT-116 Cell Line

| Cell Line | Compound 1 | Compound 2 | Compound 3 | Compound 4 | Compound 6 | Compound 7 | Compound 8 |
|---|---|---|---|---|---|---|---|
| HCT-116 (IC₅₀/ | 0.7/40.4 | 0.3/13.0 | 0.3/8.3 | 1.4/1.2 | 6.0/5.2 | 2.2/27.0 | >50 |
| SI^{a}) | | | | | | | |

^{a}SI: SI = CC₅₀ (normal cell line L-02)/IC₅₀ (tumor cell line)

As can be seen from Table 5, as far as HCT-116 cell line is concerned, Compound 8 (caerulomycin F) has no tumor cytotoxic activity against the HCT-116 cell line, while the compounds of the present disclosure have a good antitumor activity, little toxic and side effects on normal human cells, and a high safety factor, and especially Compound 2 has very good activity and selectivity against HCT-116 tumor.

TABLE 6
Cytotoxic Activity IC₅₀ (μM) and SI
of Respective Compounds to MCF-7 Cell Line

| Cell Line | Compound 2 | Compound 6 | Compound 8 |
|---|---|---|---|
| MCF-7 (IC₅₀/SI^{a}) | 1.0/3.9 | 12.0/2.6 | >50 |

^{a}SI: SI = CC₅₀(normal cell line L-02)/IC₅₀(tumor cell line)

Table 6 shows that in terms of MCF-7 cell line, Compound 8 (caemlomycin F) has no tumor cytotoxic activity against MCF-7 cell line, while the compounds of the present disclosure have a good antitumor activity, little toxic and side effects on normal human cells, and a high safety factor, and in particular Compound 2 has very good activity and selectivity against MCF-7 tumor.

TABLE 7
Cytotoxic Activity IC₅₀ (μM) and SI
of Respective Compounds to HepG2 Cell Line

| | Com- | Com- | Com- | Com- | Com- |
|---|---|---|---|---|---|
| Cell Line | pound 1 | pound 2 | pound 3 | pound 7 | pound 8 |
| HepG2 (IC₅₀/SI^{a}) | 2.7/10.5 | 0.2/19.5 | 2.0/1.3 | 10.0/6.0 | >50 |

^{a}SI: SI = CC₅₀ (normal cell line L-02)/IC₅₀ (tumor cell line)

As can be seen from Table 7 that as for HepG2 cell line, Compound 8 (caerulomycin F) has no tumor cytotoxic activity against HepG2 cell line, whereas the compounds of the present disclosure have a good antitumor activity, little toxic and side effects on normal human cells, and a high safety factor, and in particular Compound 2 has very good activity and selectivity against HepG2 tumor.

TABLE 8
Cytotoxic Activity IC₅₀ (μM) and SI of Respective Compounds to Hela Cell Line

| Cell Line | Compound 1 | Compound 2 | Compound 3 | Compound 5 | Compound 6 | Compound 7 | Compound 8 |
|---|---|---|---|---|---|---|---|
| Hela (IC₅₀/SI^{a}) | 9.0/3.1 | 0.1/39 | 1.9/1.3 | 4.4/1.7 | 19.3/1.6 | 2.5/23.8 | >50 |

^{a}SI: SI = CC₅₀ (normal cell line L-02)/IC₅₀ (tumor cell line)

It can be seen from Table 8 that as for Hela cell line, Compound 8 (caemlomycin F) has no tumor cytotoxic activity against Hela cell line, whereas the compounds of the present disclosure have a good antitumor activity, little toxic and side effects on normal human cells, and a high safety factor, and in particular Compound 2 has very good activity and selectivity against Hela tumor.

TABLE 9
Cytotoxic Activity IC₅₀ (μM) and SI of Respective Compounds to Jurkat Cell Line

| Cell Line | Compound 1 | Compound 2 | Compound 3 | Compound 6 | Compound 7 | Compound 8 |
|---|---|---|---|---|---|---|
| Jurkat (IC₅₀/SI^{a}) | 10.8/2.6 | 0.2/19.5 | 1.2/2.1 | 14.5/2.2 | 1.3/45.8 | >50 |

^{a}SI: SI = CC₅₀ (normal cell line L-02)/IC₅₀ (tumor cell line)

As can be seen from Table 9, in terms of Jurkat cell line, Compound 8 (caemlomycin F) has no tumor cytotoxic activity against Jurkat cell line, while the compounds of the present disclosure have a good antitumor activity, little toxic and side effects on normal human cells, and a high safety factor, and in particular Compound 2 has very good activity and selectivity against Jurkat tumor.

The results shown in the above Tables 4 to 9 also tell the following: (1) Compound 3 has a good in vitro antitumor activity, indicating that the tumor cytotoxic activity still remains after a hydroxyl group is substituted for the 3-methoxy group; (2) the introduction of the 3-site hydroxyl group into Compound 4 reduces the activity of the tumor cell lines and augments the toxicity to the normal cell lines, in comparison to Compound 2; (3) the introduction of 5-fluoro into Compound 5 reduces toxicity to human tumor cells and normal cells, in comparison to Compound 2; (4) compared with Compound 2, Compound 6 has a reduced toxicity to both tumor cells and normal cells after an aldehyde group is substituted for the 4-oximido group; (5) the results of Compound 1 and Compound 7 show that an oxyglucoside derivative obtained by replacing the 3-hydroxy or methoxy group with a glycosyl group remains its tumor cytotoxic activity, but the toxicity thereof is significantly reduced; (6) Compound 8 (caemlomycin F) obtained by replacing 2-pyridyl group in Compound 2 with a phenyl group has no tumor cytotoxic activity against tumor cell lines such as HCT-116, MCF-7, HepG2, Hela, and Jurkat, which indicates that an activity of a compound obtained by altering the structure of dipyridinialdoxime weakens or disappears, and this further demonstrates that the compound of formula I of the present disclosure has a good antitumor activity.

To summarize, the compound of formula I of the present disclosure has higher selectivity, lower toxicity, and a higher safety factor, while efficiently inhibiting cancer cell proliferation, and especially Compound 2 has very good antitumor activity and selectivity.

### [Test Example 2] Test for In Vivo Antitumor Activity

1. Experimental Samples and Experimental Methods

An in vivo antitumor test for Compound 2 was conducted on in vivo transplantation tumors from nude mice experiencing colon cancer HCT-116.

1. Experimental Samples and Experimental Methods

(1) Experimental Samples

Laboratory mice: BALB/C, (nu/nu) nude mice, from 18 to 20 g in weight, male; purchased from BEIJING HFK BIOSCIENCE Co., LTD.; License No.: SCXK (Jing)-2014-0004.

Positive drug: Docetaxel, dissolved with Tween DMSO as a solvent and formulated into 9 mg/mL solution for service; diluted with normal saline.

Test drug: Compound 2, 10 mg/mL of stock solution being dissolved with DMSO, diluted with normal saline till the concentration was 0.75 mg/mL, and the administration volume was 0.1 mL/10 g body weight.

(2) Experimental Methods

A cell suspension of human colon cancer cell line HCT-116 at a concentration of 30 million/mL was formulated in a 5 A medium containing 10% fetal bovine serum. After conventional disinfection, the cell suspension was inoculated subcutaneously into the armpits of the right forelimbs of the mice at a dose of 0.2 ml for each. The mice were not administered until the tumors had grown to an average volume of about 100 mm³. Positive drugs, compounds, and negative control groups were arranged. The dosages were 5 mg/kg of Docetaxel (positive), and 7.5 mg/kg of Compound 2. Routes of administration were as follows: Docetaxel was intravenously administered every other day; Compound 2 was intraperitoneally injected daily; the negative control group was intraperitoneally injected with isometric normal saline. The mice were administered for 14 days, during which the mice were weighed daily and the variation trends of body weights of the mice were calculated. The mice were killed 24 hours after the last administration. Their tumor tissues were dissected, the tumor mass was weighed, and the tumor inhibition rate was calculated. The equation for calculating the tumor inhibition rate was as follows:

tumor inhibition rate=(average tumor weight from the control group−average tumor weight from the treatment group)/average tumor weight from the control group×100%.

In the meantime, the spleens were removed from the mice, and the capsules were peeled off. The mass of the spleens of the mice was recorded respectively, and the spleen indices were calculated by the following equation:

spleen index=100×spleen mass/body mass.

Statistical analysis: The SPSS 13.0 statistical software was used for data processing. The resulting data were marked with X±S. The data from each group were analyzed by t test, and the P value was used to indicate difference between groups.

2. Experimental Results

After the administration of Compound 2 in the experiment, the behavioral observation of the mice showed that no obvious adverse effects such as allergy were observed within 14 days after administration, and the body weight of the mice was not adversely affected.

In the experiment, an average weight of the tumor in the negative control group was 1.0 g or more, which proved that the colon cancer (HCT-116) model was successfully constructed. The positive drug was a clinically broad-spectrum antitumor drug Docetaxel. In the experiment, compared with the negative control group, the 5 mg/kg Docetaxel group could significantly inhibit the growth of liver cancer (HCT-116) at an inhibition rate of 87.39% (P<0.01), indicating that the results of the experiment were credible; however, in the Docetaxel group, the body weight and spleen index of mice decreased, which reflects toxic and side effects such as inhibition of the immune system by Docetaxel.

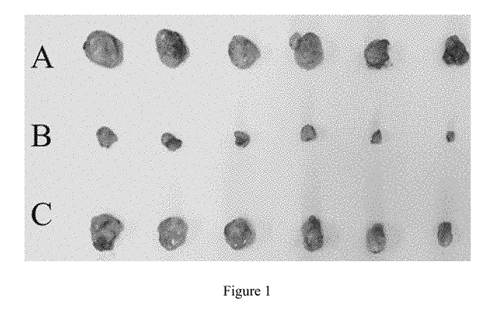

At a dose of 7.5 mg/kg for 14 days, Compound 2 had a significantly growth inhibition effect on colon cancer (HCT-116) in comparison to the negative control group (see FIG. 1, Table 10), and the inhibition rate was 42.46% (p<0.05). However, no effect of Compound 2 on the spleen index of mice was observed (see Table 11), indicating that Compound 2 was a safe and effective antitumor drug.

TABLE 10
Mass Statistics of Mouse Colon Cancer (HCT-116) (X̅ ± S unit: g)

| | Tumor Weight | Inhibition Rate % |
|---|---|---|
| Negative control (normal saline) | 1.03 ± 0.32 | / |
| Docetaxel (5 mg/kg) | 0.13 ± 0.07 | 87.39** |
| Compound 2 (7.5 mg/kg) | 0.59 ± 0.33 | 42.46* |

**p < 0.01 and *p < 0.05 represent results of comparison between each of drug administration groups and negative control group

TABLE 11
Spleen Index of Mice (spleen index =
100 × spleen mass/body mass)

| | Spleen Index |
|---|---|
| Negative control (normal saline) | 0.41 ± 0.08 |
| Docetaxel (10 mg/kg) | 0.25 ± 0.11** |
| Compound 2 (7.5 mg/kg) | 0.45 ± 0.09* |

**p < 0.01 and *p < 0.05 represent results of comparison between each of drug administration groups and negative control group

1. A compound of formula I, or pharmaceutically acceptable salt or prodrug thereof, 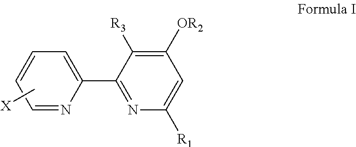
wherein,
R₁ is selected from —CH═N—O—R₄, —COR₅, —CH₂OR₆, —CN, and —NH₂;
R₄ and R₆ are each independently selected from —H, an alkyl group, and an alkanoyl group;
R₅ is selected from —H, —OH, —NH₂, and an alkoxy group;
R₂ is selected from —H, an alkyl group, and a glycosyl group;
R₃ is selected from —H, —OH, —NH₂, and an alkoxy group;
X is selected from —H, halogen, —NO₂, —SO₂R₇, and —COR₈;
R₇ is selected from —H, and an aryl group;
R₈ is an aryl group.

2. The compound of formula I, or pharmaceutically acceptable salt or prodrug thereof according to claim 1, wherein,
the alkyl group and alkyl groups from the alkanoyl group and the alkoxy group are each independently a linear or branched C₁ to C₁₆ alkyl group;
the glycosyl group is selected from: glucosyl, rhamnosyl, isorhamnosyl, ribosyl, galactosyl, allosyl, fucosyl, idosyl, talosyl, 2,4-dimethoxyrhamnosyl, 2,4-dimethoxyglucosyl, 2,4-dimethoxyisorhamnosyl, 2,4-dimethoxyribosyl, 2,4-dimethoxygalactosyl, 2,4-dimethoxyallosyl, 2,4-dimethoxyfucosyl, 2,4-dimethoxyidosyl, and 2,4-dimethoxytalosyl;
the halogen is selected from —F, —Cl, —Br, and —I;
the aryl group is a C₆ to C₁₈ monocyclic or polycyclic aryl group.

3-10. (canceled)

11. The compound of formula I, or pharmaceutically acceptable salt or prodrug thereof according to claim 2, wherein,
R₁ is selected from —CH₂OH, —CH═NOH, —CN, —CONH₂, —NH₂, —CHO, and —COOH;
R₂ is selected from —H, methyl, glucosyl, rhamnosyl, isorhamnosyl, ribosyl, galactosyl, allosyl, fucosyl, idosyl, talosyl, 2,4-dimethoxyrhamnosyl, 2,4-dimethoxyglucosyl, 2,4-dimethoxyisorhamnosyl, 2,4-dimethoxyribosyl, 2,4-dimethoxygalactosyl, 2,4-dimethoxyallosyl, 2,4-dimethoxyfucosyl, 2,4-dimethoxyidosyl, and 2,4-dimethoxytalosyl;
R₃ is selected from —H, —OH, and —OCH₃;
X is selected from —H, —F, —Cl, —Br, —I, —NO₂, —SO₃H, —SO₂C₆H₅, and —COC₆H₅.

12. The compound of formula I, or pharmaceutically acceptable salt or prodrug thereof according to claim 1, wherein the compound of formula I is Compound 1, or Compound 2, or Compound 3, or Compound 4, or Compound 5, or Compound 6,

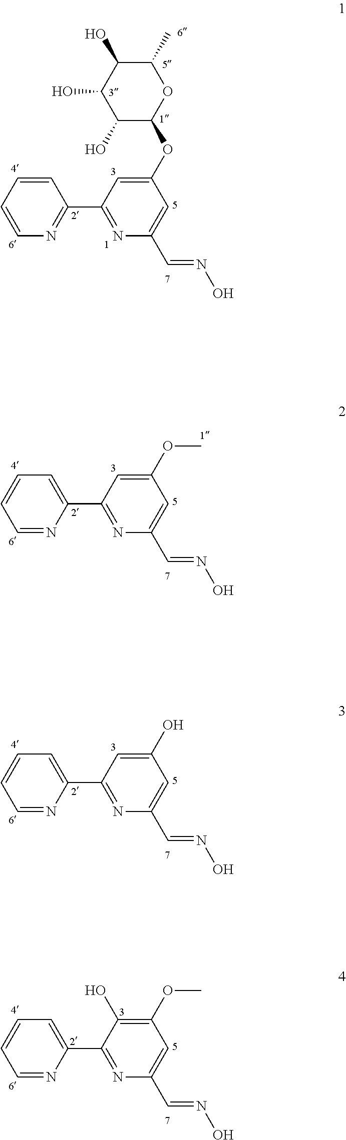 [SMILES: C[C@@H]1O[C@@H](Oc2cc(/C=N/O)nc(-c3ccccn3)c2)[C@H](O)[C@H](O)[C@H]1O ; COc1cc(/C=N/O)nc(-c2ccccn2)c1 ; O/N=C/c1cc(O)cc(-c2ccccn2)n1 ; COc1cc(/C=N/O)nc(-c2ccccn2)c1O]

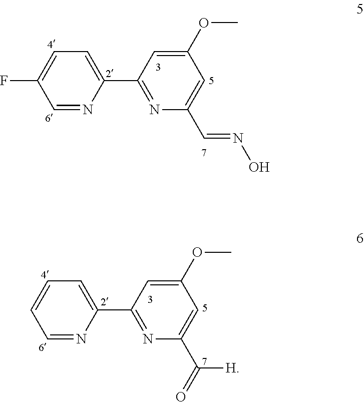

13. The compound of formula I, or pharmaceutically acceptable salt or prodrug thereof according to claim 1, wherein,
the pharmaceutically acceptable salt is a salt formed by the compound of formula I and a compound selected from the group consisting of: hydrochloric acids, sulfuric acids, phosphoric acids, formic acids, acetic acids, propionic acids, lactic acids, citric acids, tartaric acids, fumaric acids, maleic acids, mandelic acids, malic acids, and camphorsulfonic acids;
the pharmaceutically acceptable prodrug includes prodrugs formed by bonding the compound of formula I to a pharmaceutically acceptable carrier; the pharmaceutically acceptable carrier includes: triglyceride phosphate, polyethylene glycol ester, polyethylene glycol amide, or polyethylene glycol ether.

14. A method for preparing the compound of formula I, or pharmaceutically acceptable salt or prodrug thereof according to claim 1, wherein,
the compound of formula I is Compound 1, or Compound 2, or Compound 3, or Compound 4, or Compound 5, or Compound 6, or Compound 7;
the Compounds 1 to 4 are respectively obtained according to the following methods:
(a) the A. cyanogriseus WH1-2216-6 is cultured in a seed medium, inoculated to a fermentation medium to which 2-picolinic acid is added, and subjected to submerged cultivation and fermentation to obtain a fermented product; the fermented product is extracted with an organic solvent, and the organic phase is concentrated to obtain a crude extract, to which a HCl solution is added; the crude extract is extracted with an organic solvent, and the aqueous phase is adjusted to pH=8.0 with ammonium hydroxide, followed by extraction with an organic solvent, and the organic phase is concentrated to obtain an alkaloid fraction; the resulting alkaloid fraction is separated over a gel column having a mobile phase of dichloromethane:methanol=1:1 (v/v), to obtain Compound 2 and other alkaloid fractions;
(b) the other alkaloid fractions obtained in step (a) are combined and separated through silica gel column chromatography under reduced pressure by gradiently eluting with petroleum ether, dichloromethane, and dichloromethane-methanol as eluants in this order;
wherein the dichloromethane-methanol eluant includes components at ratios of: dichloromethane:methanol (v/v) being 100:1, 50:1, 30:1, 25:1, 15:1, 10:1, 8:1, 5:1, 2:1, 1:1, and 0:1;
wherein the obtained fractions from the elution by the eluant of dichloromethane and the elution by the eluant of dichloromethane:methanol=100:1 (v/v) are combined and chromatographed over a gel (Sephadex LH-20) column by eluting with dichloromethane:methanol=1:1 (v/v), and the resulting component is chromatographed over a gel (Sephadex LH-20) column by eluting with methanol to give Compound 4;
wherein the obtained fractions from the elution by the eluant of dichloromethane:methanol=8:1 (v/v) is successively undergone gel (Sephadex LH-20) column chromatography by eluting with methanol, and silica gel column flash chromatography by a gradient elution with dichloromethane and methanol to obtain 5 fractions as Fraction 1, Fraction 2, Fraction 3, Fraction 4, and Fraction 5 in the elution order; Fraction 2 is purified by semi-preparative high performance liquid chromatography (HPLC) by eluting with methanol:water=50:50 (v/v) to obtain Compound 1; Fraction 3 is purified by semi-preparative high performance liquid chromatography by eluting with methanol:water=30:70 (v/v) to obtain Compound 3;
Compound 5 is obtained according to the following method: the A. cyanogriseus WH1-2216-6 is cultured in a seed medium, inoculated into a fermentation medium to which 5-fluoropicolinic acid is added, and subjected to submerged cultivation and fermentation to obtain a fermented product; the fermented product is extracted with an organic solvent, and the organic phase is concentrated to obtain a crude extract, the crude extract is separated through silica gel column chromatography under reduced pressure by gradiently eluting with petroleum ether, dichloromethane, and dichloromethane-methanol mixture as eluants in this order; the resulting fraction is successively eluted with methanol over a gel (Sephadex LH-20) column, undergone silica gel column flash chromatography and subjected to gradient elution with dichloromethane and methanol; after concentration, the resulting fraction is purified by semi-preparative high performance liquid chromatography by eluting with methanol:water=75:25 (v/v) to obtain Compound 5;
Compound 6 is obtained by the following synthesis method: formalin, hydrochloric acid and water are added to the Compound 2 obtained by isolation, the mixture is refluxed, and then cooled to room temperature, to which a saturated aqueous solution of NaHCO₃ is added; the mixture is extracted with an organic solvent; after vacuum concentration, the organic phase is separated over a silica gel column under increased pressure by gradiently eluting with dichloromethane and methanol as eluants, to obtain Compound 6;
Compound 7 is obtained by the following method: the A. cyanogriseus WH1-2216-6 was cultured in a seed medium, inoculated into a fermentation medium, to which 2-picolinic acid was added, subjected to submerged cultivation and fermentation to obtain a fermented product; the fermented product is extracted with an organic solvent, and the organic phase is concentrated to obtain a crude extract, the crude extract is separated through silica gel column chromatography under reduced pressure by gradiently eluting with petroleum ether, dichloromethane, and dichloromethane-methanol mixed solvent (v/v, 100:1, 50:1, 30:1, 25:1, 15:1, 10:1, 5:1, 2:1, 1:1, 0:1) as eluants in this order; the obtained fraction from the elution by the eluant of dichloromethane-methanol=2:1 (v/v) is purified through semi-preparative high performance liquid chromatography by eluting with methanol:water=50:50 (v/v) to obtain Compound 7;
the organic solvent for extraction is selected from ethyl acetate, dichloromethane, chloroform, and petroleum ether.

15. A pharmaceutical composition, wherein the pharmaceutical composition comprises at least one selected from the compound of formula I, or pharmaceutically acceptable salt or prodrug thereof according to claim 1, and a pharmaceutically acceptable excipient.

16. The pharmaceutical composition according to claim 15, wherein the dosage form of the pharmaceutical composition includes an oral preparation, an injection preparation, or a transdermal preparation.

17. The pharmaceutical composition according to claim 15, wherein the dosage form of the pharmaceutical composition includes a tablet, a capsule, a powder, a granule, a lozenge, a suppository, an oral solution, a sterile parenteral suspension, or an injection.

18. The pharmaceutical composition according to claim 17, wherein the injection includes a frozen-dried powder injection.

19. Use of the compound of formula I, or pharmaceutically acceptable salts or prodrugs thereof according to claim 1 as an antitumor drug.

20. The use according to claim 19, wherein the antitumor drug is a tumor cell proliferation inhibitor or a tumor cell killer.

21. The use according to claim 20, wherein the antitumor drug is devoid of drugs against human lung adenocarcinoma A549, drugs against human acute promyelocytic leukemia HL60, drugs against human chronic myeloid leukemia K562, and drugs against human oral epidermoid carcinoma KB.

22. The use according to claim 21, wherein the antitumor drug is a drug against human colon cancer cell line HCT-116, a drug against human breast cancer cell line MCF-7, a drug against hepatoma cell line HepG2, a drug against cervical cancer cell line Hela, or a drug against human peripheral blood leukemia T cell line Jurkat.

23. Use of the pharmaceutical composition according to claim 15 as an antitumor drug.

24. The use according to claim 23, wherein the antitumor drug is a tumor cell proliferation inhibitor or a tumor cell killer.

25. The use according to claim 24, wherein the antitumor drug is devoid of drugs against human lung adenocarcinoma A549, drugs against human acute promyelocytic leukemia HL60, drugs against human chronic myeloid leukemia K562, and drugs against human oral epidermoid carcinoma KB.

26. The use according to claim 25, wherein the antitumor drug is a drug against human colon cancer cell line HCT-116, a drug against human breast cancer cell line MCF-7, a drug against hepatoma cell line HepG2, a drug against cervical cancer cell line Hela, or a drug against human peripheral blood leukemia T cell line Jurkat.

27. A probe for inhibiting cell proliferation, comprising the compound of formula I, or pharmaceutically acceptable salt or prodrug thereof according to claim 1.

28. A probe kit, wherein the probe kit comprises the probe according to claim 27.
